(19) United States
(12) Patent Application Publication
(10) Pub. No.: US 2001/0044135 A1
(43) Pub. Date: Nov. 22, 2001

(54) Chimeric molecules and novel assay system

(76) Inventors: Neil Stahi, Carmel, NY (US); Margaret Karow, Putnam Valley, NY (US); George D. Yancopoulos, Yorktown Heights, NY (US)

Correspondence Address: Linda O. Palladino, Regeneron Pharmaceuticals, Inc., 777 Old Saw Mill River Road, Tarrytown, NY, 10591, US

(21) Appl. No.: 09/821,579

(22) Filed: Mar. 29, 2001

Related U.S. Application Data
(63) Continuation of application No. 09/084,697, filed on May 26, 1998, now abandoned.

(51) Int. Cl. G01N 33/567; C07H 21/04; C12P 21/04; C12N 5/06; C07K 14/705

(52) U.S. Cl. 435/69.7; 435/7.21; 435/325; 530/350; 536/23.5

(57) ABSTRACT

The present invention provides for a method of identifying a ligand for a receptor comprising contacting a cell expressing a cell surface FcεR1 molecule with a chimeric polypeptide molecule comprising an extracellular ligand binding domain of a receptor and an IgE constant or Fc region, under conditions whereby the chimeric polypeptide molecule binds to the FcεR1 molecule to form a complex; contacting the cell bearing the complex with a ligand; and detecting or measuring ligand binding to the complex. The invention further provides for chimeric polypeptide molecules, the nucleic acids encoding the chimeric polypeptide molecules, and cell lines expressing the chimeric polypeptide molecules.

Throughout this application various publications are referenced. The disclosures of these publications in their entireties are hereby incorporated by reference into this application.

### INTRODUCTION

The field of this invention is chimeric polypeptide molecules, nucleic acid molecules encoding the chimeric polypeptide molecules, and methods of using the nucleic acid molecules and the chimeric polypeptide molecules. The present invention provides for novel assay systems useful for identifying novel ligands that interact with chimeric polypeptide molecules.

### BACKGROUND OF THE INVENTION

The ability of polypeptide ligands to bind to cells and thereby elicit a phenotypic response such as cell growth, survival, cell product secretion, or differentiation is often mediated through transmembrane receptors on the cells. The extracellular domain of such receptors (i.e. that portion of the receptor that is displayed on the surface of the cell) is generally the most distinctive portion of the molecule, as it provides the protein with its ligand binding characteristic. Binding of a ligand to the extracellular domain generally results in signal transduction which transmits a biological signal to intracellular targets. Often, this signal transduction acts via a catalytic intracellular domain. The particular array of sequence motifs of this catalytic intracellular domain determines its access to potential kinase substrates (Mohammadi, et al., 1990, Mol. Cell. Biol. 11:5068-5078; Fantl, et al., 1992, Cell 69:413-413). Examples of receptors that transduce signals via catalytic intracellular domains include the receptor tyrosine kinases (RTKs) such as the Trk family of receptors which are generally limited to cells of the nervous system, the cytokine family of receptors including the tripartate CNTF receptor complex (Stahl & Yancopoulos, 1994, J. Neurobio. 25:1454-1466) which is also generally limited to the cells of the nervous system, G-protein coupled receptors such as the β₂-adrenergic receptor found on, for instance, cardiac muscle cells, and the multimeric IgE high affinity receptor FcεRI which is localized, for the most part, on mast cells and basophils (Sutton & Gould, 1993, Nature 366:421-428).

All receptors identified so far appear to undergo dimerization, multimerization, or some related conformational change following ligand binding (Schiessinger, J., 1988, Trend Biochem. Sci. 13:443-447; Ullrich & Schlessinger, 1990, Cell 61:203-212; Schiessinger & Ullrich, 1992, Neuron 9:383-391) and molecular interactions between dimerizing intracellular domains lead to activation of catalytic function. In some instances, such as platelet-derived growth factor (PDGF), the ligand is a dimer that binds two receptor molecules (Hart, et al., 1988, Science, 240:1529-1531; Heldin, 1989, J. Biol. Chem. 264:8905-8912) while, for example, in the case of epidermal growth factor (EGF), the ligand is a monomer (Weber, et al., 1984, J. Biol. Chem. 259:14631-14636). In the case of the FcεRI receptor, the ligand, IgE, exists bound to FcεRI in a monomeric fashion and only becomes activated when antigen binds to the IgE/FcεRI complex and cross-links adjacent IgE molecules (Sutton & Gould, 1993, Nature 366:421-428).

Often, the tissue distribution of a particular receptor within higher 10 organisms provides insight into the biological function of the receptor. The RTKs for some growth and differentiation factors, such as fibroblast growth factor (FGF), are widely expressed and therefore appear to play some general role in tissue growth and maintenance. Members of the Trk RTK family (Glass & Yancopoulos, 1993, Trends in Cell Biol. 3:262-268) of receptors are more generally limited to cells of the nervous system, and the Nerve Growth Factor family consisting of nerve growth factor (NGF), brain-derived neurotrophic factor (BDNF), neurotrophin-3 (NT-3) and neurotrophin-4/5 (NT-4/5), which bind the Trk RTK family receptors, promote the differentiation of diverse groups of neurons in the brain and periphery (Lindsay, R. M, 1993, in Neurotrophic Factors, S. E. Loughlin & J. H. Fallon, eds., pp. 257-284, San Diego, Calif., Academic Press). FcεRI is localized to a very limited number of types of cells such as mast cells and basophils. Mast cells derive from bone marrow pluripotent hematopoietic stem cell lineage, but complete their maturation in the tissue following migration from the blood stream (See Janeway & Travers, 1996, in Immunobiology, 2d. Edition, M. Robertson & E. Lawrence, eds., pp. 1:3-1:4, Current Biology Ltd., London, UK, Publisher) and are involved in the allergic response.

Many studies have demonstrated that the extracellular domain of a receptor provides the specific ligand binding characteristic.

Furthermore, the cellular environment in which a receptor is expressed may influence the biological response exhibited upon binding of a ligand to the receptor. For example, when a neuronal cell expressing a Trk receptor is exposed to a neurotrophin which binds to that receptor, neuronal survival and differentiation results. When the same receptor is expressed by a fibroblast, exposure to the neurotrophin results in proliferation of the fibroblast (Glass, et al., 1991, Cell 66:405-413).

Because the identification of a large number of receptors has far exceeded the identification of their cognate ligands, many studies have attempted to take advantage of these aspects of ligand binding and the resulting phenotypic outcome in different cellular backgrounds to design assays to identify putative ligands for the receptors. In addition to the identification of putative ligands, these aspects of ligand binding and resulting phenotypic outcome have been used to characterize known ligands for relative activity (agonists) or inactivity (antagonists) and/or specificity for a given receptor. Assays commonly used which measure phenotypic changes following a ligand's binding to its receptor include phosphorylation of tyrosine residues on the intracellular domain of the receptor and/or other intracellular proteins (to measure receptor activation) or measuring DNA content or cell number as a marker for cell proliferation. While both of these assays are extremely useful, each has its own limitations and drawbacks. For instance, tyrosine phosphorylation assays are time-consuming and labor-intensive, requiring two-to-three days to complete. Another disadvantage is that these assays require the use of radioisotopes. Cell proliferation assays also generally require two-to-three days to complete and often involve either physically counting cells, performing a spectrophotometric measurement of incorporation of a reagent such as MTT or a measurement of ³H-thymidine incorporation into DNA, measuring DNA-intercalating fluorescent molecules, or a combination thereof.

Many studies designed to identify putative ligands or elucidate downstream intracellular signalling pathways have utilized chimeric receptors which contain the extracellular ligand binding domain of a receptor of interest fused to the catalytic intracellular domain of a second receptor that gives rise to a defined phenotype that is easily measured (for example, cellular proliferation). While these chimeric receptor assay systems have proven quite useful in identifying putative ligands for the receptors of interest, their primary limitation is that they are only useful for detecting ligands that bind specifically to the extracellular domain of the receptor of interest. Consequently, each newly identified receptor requires the construction of a chimeric receptor containing the extracellular domain of the newly identified receptor fused to the intracellular catalytic domain of a second receptor exhibiting a defined, easily measurable phenotype upon ligand binding. It is also generally necessary to establish a reporter cell line stably expressing the chimeric receptor. A more general, rapid assay system that could be easily modified would make ligand identification much easier.

As previously discussed, FcεRI is localized to a limited number of cell types such as mast cells and basophils. Mast cells, which mediate certain immune reactions (e.g. immediate hypersensitivity) derive from cells of bone marrow pluripotent hematopoietic stem cell lineage, but which complete their maturation in the tissue. These cells are localized in the submucosal tissue lying just beneath body surfaces, including those of the gastrointestinal and respiratory tracts, and in connective tissues along blood vessels, especially those layers known as the dermis that lie just below the skin (See Janeway & Travers, 1996, in Immunobiology, 2d. Edition, M. Robertson & E. Lawrence, eds., pp. 8:20, Current Biology Ltd., London, UK, Publisher). Basophils are similar to mast cells, but are found in the blood plasma. Both mast cells and basophils are granulated cells with prominent secretory vesicles that can be induced to release their components following antigen binding to and crosslinking of adjacent monomeric IgE molecules which are constitutively bound to FcεRI (Riske, et al., 1991, J. Biol. Chem. 266:11245-11251). This antigen-induced crosslinking of adjacent IgE/FcεRI receptor complexes causes receptor clustering and subsequent signal transduction leading to rapid secretory vesicle component release, termed degranulation. Degranulation results in the release of various mediators of the local inflammatory response, including the vasoactive amines histamine and, in some species such as mice and rabbits, serotonin (Janeway & Travers, 1996, in Immunobiology, 2d. Edition, M. Robertson & E. Lawrence, eds., pp. 8:28, Current Biology Ltd., London, UK, Publisher). For many years, histamine and other degranulation molecules have been measured as indicators of the degree of allergic response a patient is experiencing to a given antigen and to assess drug efficacy in treating disorders such as asthma (See, for example, Brown, et al., 1982, J. Allergy Clin. Immunol. 69:20-24; McBride, et al., 1989, J. Allergy Clin. Immunol. 83:374-380).

The FcεRI receptor comprises a multimeric protein complex found on the surface of mast cells and basophils, as well as eosinophils, Langerhans cells, and monocytes (Sutton & Gould, 1993, Nature 366:421-428). Clustering of the FcεRI receptor, either through crosslinking of bound monomeric IgE by multivalent antigens or by antibodies directed against the FcεRI receptor, triggers degranulation (Shimazu, et al., 1988, Proc. Natl. Acad. Sci. USA 85:1907-1911; Gilfillian, et al., 1992, J. Immuno. 149:2445-2451). Degranulation can be measured through the quantification of the released components of the secretory vesicles such as histamine, serotonin, proteases, or hexosaminidase. The FcεRI receptor is a pre-formed protein complex composed of three types of subunits (Gilfillian, et al., 1992, J. Immuno. 149:2445-2451) commonly known in the art as the a subunit (FcεRIα) (Riske, et al., 1991, J. Biol. Chem. 266:11245-11251), the β subunit (FcεRIβ) (Wilson, et al., 1995, J. Biol. Chem. 270:4013-4022), and the γ subunits (FcεRIγ) (Eiseman & Bolen, 1992, J. Biol. Chem. 267:21027-21032). The subunits exist in the stoichiometric ratio of α,β,γ₂ (Wilson, et al., 1995, J. Biol. Chem. 270:4013-4022). Each subunit is a transmembrane protein: FcεRIα has a single transmembrane domain and is solely responsible for the binding of IgE to the receptor complex (Riske, et al., 1991, J. Biol. Chem. 266:11245-11251). FcεRIβ crosses the membrane four times, with its amino terminus on the extracellular side and its carboxy terminus on the intracellular side of the membrane and appears to play a role in signal transduction (Gilfillian, et al., 1992, J. Immuno. 149:2445-2451). The two FcεRIγ subunits form disulfide-linked dimers and each subunit has a single transmembrane domain (Eiseman & Bolen, 1992, J. Biol. Chem. 267:21027-21032). The FcεRIγ subunits also appear to function in some aspects of signal transduction (Riske, et al., 1991, J. Biol. Chem. 266:11245-11251).

As noted, the extracellular domain of FcεRIα is the only portion of the FcεRI receptor complex that is required for IgE binding, and soluble versions of the FcεRI receptor composed of only the FcεRIα: extracellular domain retain the ability to bind IgE with high affinity (Kd=100 pM) (Hulett, et al., 1993, Eur. J. Immunology 23:640-645). Likewise, chimeras of FcεRIα that retain the α subunit transmembrane and cytoplasmic domains retain the ability to interact with the β subunit and γ subunits (Repetto, et al., 1996, J. of Immuno. 156:4876-4883).

The present invention provides a general, rapid assay system that uses the properties of the FcεRI receptor and its ability to induce rapid degranulation as an assay method for measuring the interaction of two substances including, but not limited to, protein:protein interactions or interactions between proteins and small organic molecules.

### SUMMARY OF THE INVENTION

The present invention provides for a general, rapid, cell-based assay system that utilizes the unique features of the IgE high affinity receptor FcεRI and the rapid, characteristic degranulation phenotype exhibited by mast cells and basophils following antigen binding to, and crosslinking of, monomeric IgE bound to the receptor on such cells.

The present invention further provides for nucleic acid molecules and the chimeric polypeptide molecules they encode comprising the extracellular ligand binding domain of a receptor and the IgE constant region; the extracellular ligand binding domain of a receptor and the IgE Fc region; a protein interacting region and the IgE constant region; or a protein interacting region and the IgE Fc region. Both the IgE constant region and the IgE Fc region have been extensively described in the literature. Briefly, the IgE constant region includes all four heavy chain constant domains (C_{H}1-C_{H}4) while the IgE Fc region corresponds to three of the four heavy chain constant domains (C_{H}2-C_{H}4) (See Janeway & Travers, 1996, in Immunobiology, 2d. Edition, M. Robertson & E. Lawrence, eds., pp. 3:1-3:39, Current Biology Ltd., London, UK, Publisher). A protein interacting region, also called a protein module or protein binding domain, is defined as protein or peptide sequences or motifs that, in their native state, are located on proteins that are found in the intracellular environment. Such intracellular proteins, containing protein interacting regions, interact with various intracellular targets, such as kinases, phosphatases, or the intracellular domains of receptors, and can signal any one of a number of different cellular responses including, but not limited to, gene expression, cytoskeletal architecture, protein trafficking, adhesion, migration, and metabolism.

In particular, the chimeric polypeptide molecules comprise an extracellular ligand binding domain selected from a group consisting of the granulocyte colony stimulating factor (“GCSF”) receptor extracellular ligand binding domain, the muscle-specific kinase (“MuSK”) receptor extracellular ligand binding domain, the bone morphogenic protein (“BMP”) receptor extracellular ligand binding domain, the leptin (“Ob”) receptor extracellular ligand binding domain, the ciliary neurotrophic factor receptor alpha (“CNTFRα”) extracellular ligand binding domain, the gp130 receptor extracellular ligand binding domain, and the erythopoietin (“EPO”) receptor extracellular ligand binding domain.

In other embodiments, the chimeric polypeptide molecules comprise a protein interacting region selected from among the src homology 2 (“SH2”) domain, the src homology 3 (“SH3”) domain, the postsynaptic density protein/discs-large protein/zonula occludens-1 (PDZ) domain (also known as the DHR domain and GLGF repeats), the janus-associated kinase (“JAK”) binding domain, the PH domain, and the WW domain.

In a further aspect, the present invention provides an assay system comprising a chimeric polypeptide molecule comprising an extracellular ligand binding domain of a receptor fused to an IgE constant region and a means of detecting or measuring ligand binding to the chimeric polypeptide molecule.

In a further aspect, the present invention provides an assay system comprising a chimeric polypeptide molecule comprising an extracellular ligand binding domain of a receptor fused to an IgE Fc region and a means of detecting or measuring ligand binding to the chimeric polypeptide molecule. The present invention provides an assay system comprising a chimeric polypeptide molecule comprising a protein interacting region fused to an IgE constant region or an IgE Fc region and a means of detecting or measuring ligand binding to the chimeric polypeptide molecule. The means of detecting or measuring ligand binding to the chimeric polypeptide molecule comprises detecting or measuring dimerization of a complex of the chimeric polypeptide molecule and a cell surface FcεRI molecule resulting from ligand binding to the chimeric polypeptide molecule. Dimerization of the complex can involve cellular degranulation resulting from dimerization and may be detected or measured by a colorimetric assay, a radioisotopic assay, or a fluorescence assay.

In particular, the present invention provides an assay system wherein the extracellular ligand binding domain of the receptor is selected from among the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, and the EPO receptor extracellular ligand binding domain.

In a further aspect, the present invention provides an assay system wherein the ligand that binds the chimeric polypeptide molecule may be a protein, a peptide, a lipid, a carbohydrate, a nucleic acid, or a small molecule.

The present invention also provides cell lines that stably express chimeric polypeptide molecules comprising an extracellular ligand binding domain of a receptor and an IgE constant region; or comprising an extracellular ligand binding domain of a receptor and an IgE Fc region; or comprising a protein interacting region and an IgE constant region; or comprising a protein interacting region and an IgE Fc region.

In a particular embodiment, the present invention provides cell lines wherein the extracellular ligand binding domain of a receptor of the chimeric polypeptide molecule produced by the cell lines may be the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, or the EPO receptor extracellular ligand binding domain.

In another particular aspect, the chimeric protein produced by the cell lines comprise a protein interacting region that is selected from among the SH2 domain, the SH3 domain, the PDZ domain, the JAK binding domain, the PH domain, and the WW domain.

The present invention provides a method of identifying a ligand for a receptor comprising contacting a cell expressing a cell surface FcεR1 molecule with a chimeric polypeptide molecule comprising an extracellular ligand binding domain of a receptor and an IgE constant region or an IgE Fc region, under conditions whereby the chimeric polypeptide molecule binds to the FcεR1 molecule to form a complex therewith; contacting the cell bearing said complex with a ligand, wherein ligand binding may be detected or measured by detecting or measuring dimerization of the complexes, wherein dimerization is indicative of the identification of a ligand for the receptor.

In a particular embodiment, the cell expressing the cell surface FcεR1 molecule is a mast cell or a basophil.

In particular, dimerization of the complex may be detected or measured by cellular degranulation resulting from dimerization wherein cellular degranulation may be detected or measured by a colorimetric assay, a radioisotopic assay, or a fluorescence assay.

In a particular embodiment, the extracellular ligand binding domain of the method may be the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, or the EPO receptor extracellular ligand binding domain.

In a further aspect of the method, the ligand that binds the chimeric polypeptide molecule may be a protein, peptide, lipid, carbohydrate, nucleic acid, or a small molecule.

The present invention also provides a method of identifying an antagonist to a receptor wherein the method comprises contacting a cell expressing a cell surface FcεR1 molecule with (i) a chimeric polypeptide molecule comprising an extracellular ligand binding domain of a receptor and an IgE constant region or an IgE Fc region; and (ii) a ligand for the receptor, in the presence and absence of an antagonist for the receptor, and under conditions whereby the chimeric polypeptide molecule binds to the cell surface FcεR1 molecule to form a complex therewith; and (iii) measuring binding of the ligand to the complex in the presence and absence of the antagonist, wherein decreased ligand binding in the presence of the candidate antagonist is indicative of identification of an antagonist.

In a particular embodiment of the method, the cell expressing a cell surface FcεR1 molecule is a mast cell or a basophil.

In particular, the method involves detecting or measuring dimerization of the complex resulting from ligand binding thereto, wherein dimerization may be detected or measured by cellular degranulation resulting from dimerization. Cellular degranulation may be detected or measured by a calorimetric assay, a radioisotopic assay, or a fluorescence assay.

In particular embodiments, the extracellular ligand binding domain of the method may be the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, or the EPO receptor extracellular ligand binding domain.

In another aspect, the ligand that binds the chimeric polypeptide molecule may be a protein, peptide, lipid, carbohydrate, nucleic acid, or a small molecule.

The present invention also provides a method of making a chimeric polypeptide molecule comprising an extracellular binding domain of a receptor and an IgE constant region or an IgE Fc region comprising introducing a nucleic acid molecule encoding an extracellular binding domain of a receptor and an IgE constant region or an IgE Fc region into a host cell, maintaining the host cell under conditions whereby the nucleic acid is expressed to produce a chimeric polypeptide molecule, and recovering the chimeric polypeptide molecule in purified form.

The present invention additionally provides a method of making a chimeric polypeptide molecule comprising a protein interacting region and an IgE constant region or an IgE Fc region comprising introducing a nucleic acid molecule encoding a protein interacting region and an IgE constant region or an IgE Fc region into a host cell, maintaining the host cell under conditions whereby the nucleic acid is expressed to provide a chimeric polypeptide molecule, and recovering the chimeric polypeptide molecule in purified form.

The present invention further provides the method wherein the host 10 cell may be a bacterial cell, a yeast cell, an insect cell, or a mammalian cell.

The present invention further provides vectors comprising (i) a nucleic acid molecule encoding an extracellular ligand binding domain of a receptor and an IgE constant region; (ii) an extracellular ligand binding domain of a receptor and an IgE Fc region; (iii) a protein interacting region and an IgE constant region; or (iv) a protein interacting region and an IgE Fc region.

In a particular embodiment, the present invention provides nucleic acid molecules wherein the extracellular ligand binding domain of the receptor encoded thereby may be the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, or the EPO receptor extracellular ligand binding domain.

In a further embodiment, the present invention provides nucleic acid molecules wherein the protein interacting region encoded thereby may be the SH2 domain, the SH3 domain, the PDZ domain, the JAK binding domain, the PH domain, or the WW domain.

### DETAILED DESCRIPTION OF THE INVENTION

The present invention provides for a general, rapid, cell-based assay that utilizes the unique features of the IgE high affinity receptor FcεRI which is found on mast cells and basophils and the rapid, characteristic degranulation phenotype exhibited by these cells following antigen binding to and crosslinking of bound monomeric IgE and the subsequent dimerization of FcεR1 receptors. Although many receptors, as well as other secreted or cell surface proteins, have been produced as chimeric polypeptide molecules comprising an extracellular ligand binding domain or other domain of interest to either the constant region or the Fc region of IgG or IgM, there are no reported examples of chimeric polypeptide molecules in which an extracellular ligand binding domain or other domain of interest has been fused to an IgE constant region or an IgE Fc region. Thus, one feature of the present invention provides chimeric polypeptide molecules useful in a general, rapid, cell-based assay aimed at identifying ligands to receptors of interest or agents that interact with other protein domains of interest.

Chimeric polypeptide molecules are made by fusing two different polypeptide molecules into one polypeptide molecule. Many uses for chimeric polypeptide molecules have been reported in the literature. For example, chimeric polypeptide molecules that are a fusion between the extracellular ligand binding domain of a cell surface receptor fused to the IgG Fc region molecule have been used to identify unknown ligands for receptors or to block endogenous ligand from binding to its receptor (See, for example, Goodwin, et. al., 1993, Cell 73:447-456). Chimeric polypeptide molecules also include a polypeptide molecule of interest fused to a short polypeptide “tag” that is generally only a few amino acids long. One such example is the histidine (HIS) tag which is composed of six histidine residues, and is generally fused to the carboxy terminus of the polypeptide of interest. HIS-tagged chimeric polypeptide molecules may be readily purified from, for example, culture media using affinity chromatography wherein a metal such as nickel or cobalt has been immobilized on a solid support and the HIS tagged proteins binds to the metal. Passage of the culture media containing the HIS-tagged chimeric polypeptide molecule over such solid support effectively purifies the HIS-tagged chimeric polypeptide molecule from the culture media.

The present invention provides for unique chimeric polypeptide molecules formed by fusing either an extracellular ligand binding domain of a cell surface receptor of interest or some other protein interacting region of interest to either an IgE constant region or an IgE Fc region that are suitable for use in a rapid cell-based assay.

An extracellular ligand binding domain is defined as the portion of a receptor that, in its native conformation in the cell membrane, is oriented extracellularly where it can contact with its cognate ligand. The extracellular ligand binding domain does not include the hydrophobic amino acids associated with the receptor's transmembrane domain or any amino acids associated with the receptor's intracellular domain. Generally, the intracellular or cytoplasmic domain of a receptor is usually composed of positively charged or polar amino acids (i.e. lysine, arginine, histidine, glutamic acid, aspartic acid). The preceding 15-30, predominantly hydrophobic or apolar amino acids (i.e. leucine, valine, isoleucine, and phenylalanine) comprise the transmembrane domain. The extracellular domain comprises the amino acids that precede the hydrophobic transmembrane stretch of amino acids. Usually the transmembrane domain is flanked by positively charged or polar amino acids such as lysine or arginine. von Heijne has published detailed rules that are commonly referred to by skilled artisans when determining which amino acids of a given receptor belong to the extracellular, transmembrane, or intracellular domains (See von Heijne, 1995, BioEssays 17:25-30). Alternatively, websites on the Internet, such as http://ulrec3.unil.ch/software/ TMPRED_form.html. have become available to provide protein chemists with information about making predictions about protein domains.

Examples of receptors whose extracellular ligand binding domain is useful in the chimeric polypeptides of the present invention include, inter alia, the granulocyte colony-stimulating factor receptor (GCSFR), the muscle-specific kinase (MuSK) receptor, the leptin receptor (Ob-R), the CNTFRA receptor component, the gp130 receptor component, and the erythropoietin receptor (EPOR), each of which is described below. GCSFR is found on granulocytes (Ito, et al., 1994, Eur J. Biochem. 220:881-891) and non-hematopoietic cells including endothelial cells and melanoma cells (Baldwin, et al., 1991, Blood 78:609-615) and binds the ligand granulocyte colony-stimulating factor (GCSF) which induces differentiation of granulocytes into neutrophils. In accordance with the present invention, GCSFR extracellular ligand binding domain/IgE constant region or IgE Fc region chimeric polypeptide molecules are constructed for use in, for example, a high throughput assay designed to screen for agonists of GCSFR. GCSFR agonists are useful in increasing neutrophil numbers in an individual whose neutrophil count has been reduced, a condition known as neutropenia, resulting from cancer chemotherapy or other treatment regimens or diseases which result in a decrease in neutrophil cell count. In order to construct a chimeric polypeptide molecule comprising GCSFR, the GCSFR extracellular ligand binding domain DNA is PCR-amplified by standard techniques using a Human Bone Marrow cDNA Library (Clontech catalog #HL5005a) as a PCR template. The DNA encoding an IgE constant region or IgE Fc region is PCR-amplified by standard techniques using a Balbic Mouse Spleen cDNA Library (Clontech catalog #ML5011t). The resulting PCR-amplified DNA fragments are fused together by standard recombinant DNA techniques, placed into a suitable vector under the control of expression control sequences (i.e. promoters and enhancers) which is then introduced into a suitable host for production of the recombinant chimeric polypeptide molecules (See Sambrook, et al., Molecular Cloning, A Laboratory Manual, Cold Spring Harbor Laboratory; Current Protocols in Molecular Biology, Eds. Ausubel, et al., Greene Publ. Assoc., Wiley-lnterscience, NY).

MuSK is localized at the motor end plate region of adult muscle cells and, in concert with its ligand agrin and an accessory receptor protein known as MASC, is important in the formation of the neuromuscular junction (Glass, et al., 1997, Proc. Natl. Acad. Sci. USA 44:8848-8853).

MuSK expression is known to be significantly upregulated under conditions of muscle atrophy and denervation (Valenzuela, et al., 1995, Neuron 15:573-584). In accordance with the present invention, a MuSK extracellular ligand binding domain/IgE constant region or IgE Fc region chimeric polypeptide molecule is constructed for use in, for example, a high throughput assay to screen for agonists of MuSK that are useful in alleviating or reducing muscle atrophy resulting from disease, disuse or denervation. In order to construct a chimeric polypeptide molecule comprising a MuSK extracellular ligand binding domain, DNA encoding the MuSK extracellular domain is PCR-amplified by standard techniques using a Human Skeletal Muscle cDNA Library (Clontech catalog #HL5023) as a PCR template. DNA encoding the IgE constant region or IgE Fc region is PCR-amplified by standard techniques using a Balb/c Mouse Spleen cDNA library (Clontech catalog #ML5011t). The resulting PCR-amplified DNA fragments are fused together by standard recombinant DNA techniques, placed into a suitable vector under the control of expression control sequences (i.e. promoters and enhancers) which is then introduced into a suitable host for production of the recombinant chimeric polypeptide molecules (See Sambrook, et al., Molecular Cloning, A Laboratory Manual, Cold Spring Harbor Laboratory; Current Protocols in Molecular Biology, Eds. Ausubel, et al., Greene PubI. Assoc., Wiley-Interscience, NY).

The Ob-R is highly expressed in the hypothalamus, the brain center responsible for regulating food intake and satiety (Spanswick, et al., 1997, Nature 390:521-525). Leptin, the ligand for Ob-R, is known to decrease food intake when bound to Ob-R. In accordance with the present invention, an Ob-R extracellular ligand binding domain/lgE constant region or IgE Fc region chimeric polypeptide molecule is constructed for use in, for example, a high throughput assay to screen for agonists or antagonists of Ob-R. Agonists could signal a decrease in food intake and thus be useful in the treatment of obesity. Antagonists might be useful in the treatment of, for example, anorexia or cachexia by stimulating food intake. To construct such a chimeric polypeptide molecule, the Ob-R extracellular ligand binding domain DNA is PCR-amplified by standard techniques using a Human Brain, Hypothalamus cDNA Library (Clontech catalog #HL1172a) as a PCR template. The IgE constant region or IgE Fc region is PCR-amplified by standard techniques using a Balb/c Mouse Spleen cDNA Library (Clontech catalog #ML5011t). The resulting PCR-amplified DNA fragments are fused together by standard recombinant DNA techniques, placed into a suitable vector under the control of expression control sequences (i.e. promoters and enhancers) which is then introduced into a suitable host for production of the recombinant chimeric polypeptide molecules (See Sambrook, et al., Molecular Cloning, A Laboratory Manual, Cold Spring Harbor Laboratory; Current Protocols in Molecular Biology, Eds. Ausubel, et al., Greene PubI. Assoc., Wiley-lnterscience, NY).

The EPOR is expressed on erythroblast cells and binds the ligand erythropoietin (Liboli, et al, 1993, PNAS USA 90:11351-11355). Binding of erythropoietin on these cells stimulates red blood cell differentiation and proliferation. The present invention provides for chimeric polypeptide molecules comprising the extracellular ligand binding domain of EPOR fused to either the IgE constant region or IgE Fc region which is constructed for use in assays to screen for agonists or antagonists of EPOR. An agonist of EPOR would be useful in treating patients undergoing cancer chemotherapy and whose red blood cell counts are diminished, a condition known as erythropenia. An antagonist of EPOR would be useful to treat erythrocythemia, a condition characterized by too many erythrocytes. To construct such a chimeric polypeptide molecule, the EPOR extracellular ligand binding domain DNA is PCR-amplified by standard techniques using a Human Bone Marrow cDNA Library (Clontech catalog #HL5005a) as a PCR template. The IgE constant region or IgE Fc region is PCR-amplified by standard techniques using a Balb/c Mouse Spleen cDNA Library (Clontech catalog #ML5011t). The resulting PCR-amplified DNA fragments are fused together by standard recombinant DNA techniques, placed into a suitable vector under the control of expression control sequences (i.e. promoters and enhancers) which is then introduced into a suitable host for production of the recombinant chimeric polypeptide molecules (See Sambrook, et al., Molecular Cloning, A Laboratory Manual, Cold Spring Harbor Laboratory; Current Protocols in Molecular Biology, Eds. Ausubel, et al., Greene Publ. Assoc., Wiley-Interscience, NY).

Both CNTFRα and gp130 are receptor components that are used by a number of cytokine receptors including, but not limited to, ciliary neurotrophic factor (CNTF), leukemia inhibitory factor (LIF), interleukin-6 (IL6), and oncostatin M (OSM) receptors (Stahl and Yancopoulos, 1993, Cell 74:587-590). Chimeric polypeptide molecules comprising the extracellular ligand binding domain of either of these two receptor components fused to an IgE constant region or IgE Fc region are constructed for use in assays screening for either agonists or antagonists of the CNTF, LIF, IL6, or OSM receptors. Both the CNTFRα and gp130 extracellular ligand binding domain DNAs can be PCRamplified by standard techniques using a Human Skeletal Muscle cDNA Library (Clontech catalog #HL5023) as a PCR template. The IgE constant region or IgE Fc region can be PCR-amplified by standard techniques using a Balb/c Mouse Spleen cDNA Library (Clontech catalog #ML5011t). The resulting PCR-amplified DNA fragments are fused together by standard recombinant DNA techniques, placed into a suitable vector under the control of expression control sequences (i.e. promoters and enhancers) which is then introduced into a suitable host for production of the recombinant chimeric polypeptide molecules (See Sambrook, et al., Molecular Cloning, A Laboratory Manual, Cold Spring Harbor Laboratory; Current Protocols in Molecular Biology, Eds. Ausubel, et al., Greene Publ. Assoc., Wiley-lnterscience, NY).

As stated previously, a protein interacting region, also called a protein module or protein binding domain, is defined as protein or peptide sequences or motifs that, in their native state, are located on proteins that are found in the intracellular environment. Such intracellular proteins, containing protein interacting regions, interact with various intracellular targets, such as kinases, phosphatases, or the intracellular domains of receptors, and can signal any one of a number of different cellular responses including, but not limited to, gene expression, cytoskeletal architecture, protein trafficking, adhesion, migration, and metabolism. Several non-limiting examples of well known protein interacting regions that have been extensively reported in the literature include the Src homology 2 (SH2) domain, the Src homology 3 (SH3) domain, the postsynaptic density protein/discs-large protein/zonula occludens-1 (PDZ) domain (also known as the DHR domain and GLGF repeats), the janus-associated kinase (JAK) binding domain, the PH domain, and the WW domain, all of which are described below.

SH2 and SH3 each recognize short peptide motifs that contain either phosphotyrosine (SH2) or one or more proline residues (SH3) with the consensus sequence Pro-X-X-Pro (See Pawson, 1995, Nature 373:573580; Cohen, et al., 1995, Cell 80:237-248). PDZ consists of a 90 amino acid residue repeat found in a number of proteins implicated in ion-channel and receptor clustering (See, for example, Cabral, et al., 1996, Nature 382:649-652). Some PDZ domains are protein interacting regions that recognize the consensus carboxy-terminal tripeptide motif Ser/Thr-X-Val with high specificity. The JAK binding domains are protein interacting region motifs that are found on a family of nontyrosine kinases, the Janus kinases (See Cohen, et al., 1995, Cell 80:237-248). They associate with membrane bound receptors, for example platelet-derived growth factor receptor (PDGFR), and, when activated, phosphorylate members of the STAT family. The PH domain is a region of approximately 100 amino acids that is found on a wide variety of signaling and cytoskeletal proteins (See Cohen, et al., 1995, Cell 80:237-248). PH domains are somewhat different from other protein interacting regions in that the similarity between them is not at the amino acid level, where the homology is relatively low, but rather at the protein folding level, where they are virtually identical (See Cohen, et al., 1995, Cell 80:237-248). The WW domain is present in a number of different signalling and regulatory proteins and recognizes ligands that contain Pro-rich regions, some of which have the core consensus sequence X-Pro-Pro-X-Tyr (See, for example, Einbond & Sudol, 1996, FEBS Letters 384:1-8).

Nucleic acid molecules encoding chimeric polypeptide molecules comprising the protein interacting regions SH2, SH3, PDZ, JAK, PH, or WW, or any other protein interacting region, fused to either the IgE constant region or IgE Fc region, may be constructed as set forth above. However, because protein interacting regions are normally found on intracellular proteins, they do not contain signal peptide sequences that direct secretion or translocation across the cell membrane. Therefore, the construction of chimeric polypeptide molecules comprising a protein interacting region that can be secreted or translocated across the cell membrane requires inclusion of a signal peptide sequence, such as an IgK signal peptide, at the 5′ end of the protein interacting region to accomplish translocation of the chimeric polypeptide molecule (See, for example, Chaudhary, et al., 1997, Immunity 7:821-830).

In accordance with the present invention, the chimeric polypeptide molecules are used in a cell-based assay to screen for agents that interact with a protein interacting region of the chimeric polypeptide molecules. By way of a non-limiting example, the purified chimeric polypeptide molecule is bound to the FcεR1 receptor on mast cells, basophils, or an appropriate cell line, and then contacted with a test sample such as a cell lysate, conditioned cell culture media, or a small molecule library to assay for agents that interact with a protein interacting region of the chimeric polypeptide molecule. Binding of an agent in the test sample induces degranulation of the mast cell, basophil, or another appropriate cell that is able to degranulate.

Degranulation, which occurs after the FceR1 receptor is crosslinked (usually as a result of antigen binding to IgE), is characterized by the exocytotic release by mast cells and basophils of several different mediators of inflammation including, inter alia, histamine, serotonin, tryptase, β-hexosaminidase, β-glucuronidase, and arachidonic acid. Assays to detect or measure the release of any one or several of these substances may be readily performed to monitor degranulation. Assays include, but are not limited to, calorimetric assays (See, for example, Wenzel, et al., 1986, J. Immunol. Methods 86:139-142; Lavens, et al., 1993, J. Immunol. Methods 166:93-102; Schwartz, et al., 1981, J. of Immunol. 126:1290-1294; Schwartz, et al., 1979, J. of Immunol. 123:1445-1450), radioisotopic assays (See, for example, Mazingue, et al., 1978, J. Immunol. Methods 21:65-77; Brown, et al., 1982, J. Allergy Clin. Immunol. 69:20-24; Hirasawa, et al., J. of Immunol. 154:53915402), or fluorescent assays (See, for example, Kawasaki, et al., 1991, Biochim. Biophys. ACTA 1067:71-80; Furuno, et al., 1992, Immunol. Lett. 33:285-288; MacGlashan, D. W., Jr., 1995, J. Leukoc. Biol. 58:177-188).

Both the IgE constant region and the IgE Fc region have been extensively described in the literature. Briefly, the IgE constant region includes all four heavy chain constant domains (C_{H}1-C_{H}4) while the IgE Fc region corresponds to three of the four heavy chain constant domains (C_{H}2-C_{H}4) (See Janeway & Travers, 1996, in Immunobiology, 2d. Edition, M. Robertson & E. Lawrence, eds., pp. 3:1-3:39, Current Biology Ltd., London, UK, Publisher). One skilled in the art would be able to refer to any of a number of publications (See, for example, Shinkai, et al., 1988, Immunogenetics 27:288-292) to determine the exact amino acid residues in the IgE constant or IgE Fc region that should be included in the construction of chimeric polypeptide molecules of the disclosed invention. Briefly, IgE constant region-containing chimeric polypeptide molecules constructed according to the present invention would have at the 5′ fusion site the DNA sequence: 5′TGATTA-GCCCGGGC-TCTATCAGGAACCCTCAGCTCTACC3′. This DNA sequence corresponds to six nonsense nucleotides (underlined), an eight nucleotide Srfl cloning site (italicized), and the nucleotides encoding first eight amino acids of the IgE C_{H}1 domain (Ser-Ile-Arg-Asn-Pro-Gln-Leu-Tyr).

The IgE Fc region-containing chimeric polypeptide molecules constructed according to the present invention would have at the 5′ fusion site the DNA sequence: 5′TGTCTAG-GCCCGGGC-CGACTGTCAACATCACTGAGCC3′. This DNA sequence corresponds to seven nonsense nucleotides (underlined), an eight nucleotide Srfl cloning site (italicized), and the nucleotides encoding the second through eighth amino acids of the IgE C_{H}2 domain (Arg-Pro-Val-Asn-lle-Thr-Glu-Pro).

The 3′ end of both the IgE constant region-containing and the IgE Fc region-containing chimeric polypeptide molecules constructed according to the present invention would terminate with the following DNA sequence: 5′GAACTA-GCGGCCGC-CTAGGAGGGACGGAGGGAGGTG3′. This DNA sequence corresponds to a six nucleotide nonsense sequence (underlined), an eight nucleotide NotI cloning site (italicized), and the nucleotides encoding the last six amino acids and stop codon of the IgE C_{H}4 domain.

The present invention provides for the construction of nucleic acid molecules encoding chimeric polypeptide molecules that are inserted into a vector that is able to express the chimeric polypeptide molecules when introduced into an appropriate host cell. Appropriate host cells include, but are not limited to, bacterial cells, yeast cells, insect cells, and mammalian cells. Any of the methods known to one skilled in the art for the insertion of DNA fragments into a vector may be used to construct expression vectors encoding the chimeric polypeptide molecules under control of transcriptional/translational control signals. These methods may include in vitro recombinant DNA and synthetic techniques and in vivo recombinations (genetic recombination) (See Sambrook, et al., Molecular Cloning, A Laboratory Manual, Cold Spring Harbor Laboratory; Current Protocols in Molecular Biology, Eds. Ausubel, et al., Greene Publ. Assoc., Wiley-lnterscience, NY).

Expression of nucleic acid molecules encoding the chimeric polypeptide molecules may be regulated by a second nucleic acid sequence so that the chimeric polypeptide molecule is expressed in a host transformed with the recombinant DNA molecule. For example, expression of the chimeric polypeptide molecules described herein may be controlled by any promoter/enhancer element known in the art. Promoters which may be used to control expression of the chimeric polypeptide molecules include, but are not limited to, the long terminal repeat as described in Squinto et al., (1991, Cell 65:1-20); the SV40 early promoter region (Bernoist and Chambon, 1981, Nature 290:304-310), the CMV promoter, the M-MuLV 5′ terminal repeat the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad., Sci. U.S.A. 78:144-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25, see also “Useful proteins from recombinant bacteria” in Scientific American, 1980, 242:74-94); promoter elements from yeast or other fungi such as the Gal 4 promoter, the ADH (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58); alpha 1-antitrypsin gene control region which is active in the liver (Kelsey et al, 1987, Genes and Devel. 1:161171), beta-globin gene control region which is active in myeloid cells (Mogram et al., 1985, Nature 315:338-340; Kollias et al., 1986, Cell 46:89-94); myelin basic protein gene control region which is active in oligodendrocyte cells in the brain (Readhead et al., 1987, Cell 48:703-712); myosin light chain-2 gene control region which is active in skeletal muscle (Shani, 1985, Nature 314:283-286), and gonadotropic releasing hormone gene control region which is active in the hypothalamus (Mason et al., 1986, Science 234:1372-1378).

By way of non-limiting example, a nucleic acid encoding a chimeric polypeptide molecule may be constructed that contains an IgE constant region fused to an extracellular domain of a receptor of interest, for instance, a RTK. This nucleic acid is inserted into a vector under the control of a promoter and other expression control sequences which is used to transfect a cell line, where the nucleic acid is expressed and chimeric polypeptide molecules are secreted into the media, from which they are purified by any number of techniques known to one skilled in the art. This purified chimeric polypeptide molecule is bound to the FcεR1 receptor on mast cells, basophils, or an appropriate cell line able to degranulate, and then contacted with a test sample such as a cell lysate or conditioned cell culture media, to assay for putative ligands. Binding of a putative ligand in the test sample induces degranulation of cells. A second use of the secreted chimeric polypeptide molecule is in a competitive assay wherein the test sample containing the putative ligand is exposed to a cell line expressing the FcεR1 receptor, and the chimeric polypeptide molecule is added in varying concentrations to determine whether ligand binding may be competed for.

The ligands may be agonists, in that they elicit a biological response following binding to their cognate receptor, or antagonists, which can either prevent a biological response upon binding, or, as has recently been reported for Angiopoietin1/Angiopoietin2 by Maisonpierre, et al., 1997, Science 277:55-60, act as a naturally occurring antagonist for a known agonist. The assay system of the present invention may be adapted readily to screen for ligands for many different receptors, and is extremely useful for both laboratory-scale ligand identification as well as high throughput screening of small molecule libraries to identify receptor agonists or antagonists.

The ligands may include, but are not limited to, a protein, a peptide or polypeptide, a lipid, a carbohydrate, a nucleic acid, or a small molecule, preferably a small organic molecule, and are obtained from a wide variety of sources including, but not limited to, libraries of synthetic or natural compounds. Novel ligands that bind to the chimeric polypeptide molecules described herein may mediate degranulation in cells naturally expressing the FcεR1 receptor, but also may confer a degranulation phenotype when used to treat cells engineered to express the FcεR1 receptor.

Thus, according to the invention, expression vectors capable of being replicated in a bacterial or eukaryotic host comprising chimeric polypeptide molecule-encoding nucleic acid as described herein, are used to transfect the host and thereby direct expression of such nucleic acids to produce the chimeric polypeptide molecules, which may then be recovered in a biologically active form. As used herein, a biologically active form includes a form capable of binding to the FcεR1 receptor and mediating degranulation.

Expression vectors containing the chimeric nucleic acid molecules described herein can be identified by three general approaches: (a) DNA-DNA hybridization, (b) presence or absence of “marker” gene functions, and (c) expression of inserted sequences. In the first approach, the presence of a foreign gene inserted in an expression vector can be detected by DNA-DNA hybridization using probes comprising sequences that are homologous to the inserted chimeric polypeptide molecule sequences. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign genes in the vector. For example, if the chirneric polypeptide molecule DNA sequence is inserted within the marker gene sequence of the vector, recombinants containing the insert can be identified by the absence of the marker gene function. In the third approach, recombinant expression vectors can be identified by assaying the foreign gene product expressed by the recombinant. Such assays can be based, for example, on the physical or functional properties of the chimeric polypeptide molecules, for example, by binding to the FcεR1 receptor and mediating degranulation.

Cells of the present invention may transiently or, preferably, constitutively and permanently express the chimeric polypeptide molecules.

The chimeric polypeptide molecules may be purified by any technique which allows for the subsequent formation of a stable, biologically active chimeric polypeptide molecule. For example, and not by way of limitation, the factors may be recovered from cells either as soluble proteins or as inclusion bodies, from which they may be extracted quantitatively by 8M guanidinium hydrochloride and dialysis (see, for example, Builder, et al., U.S. Pat. No. 5,663,304). In order to further purify the factors, conventional ion exchange chromatography, hydrophobic interaction chromatography, reverse phase chromatography or gel filtration may be used.

The present invention also has diagnostic and therapeutic utilities. In particular embodiments of the invention, methods of detecting aberrancies in the function or expression of the chimeric polypeptide molecules described herein may be used in the diagnosis of disorders.

In other embodiments, manipulation of the chimeric polypeptide molecules or agonists or antagonists which bind the chimeric polypeptide molecules may be used in the treatment of diseases. In further embodiments, the chimeric polypeptide molecule is utilized as an agent to block the binding of a binding agent to its target.

The following examples are offered by way of illustration and not by way of limitation.

### EXAMPLES

### Example 1

### Construction of Chimeric IgE Molecules

A chimeric DNA molecule was constructed encoding a fusion protein containing the extracellular domain of the granulocyte-colony stimulating factor receptor (GCSFR) and the constant region of the mouse IgE heavy chain.

This chimeric DNA molecule was constructed as follows: DNA encoding the four IgE constant region domains, termed C_{H}1-C_{H}4, were PCR-amplified from Balb/C mouse spleen cDNA (Clonetech) using an IgE 5′ (constant) primer with the sequence 5′TGATTAGCCCGGGCTCTATCAGGAACCCTCAGCTCT ACC3′ and an IgE 3′ primer with the sequence 5′GAACTAGCGGCC GCCTAGGAGGGACGGAGGGAGGTG3′. The IgE 3′ primer contained a NotI restriction site 3′ of the translational stop codon for cloning purposes. The IgE 5′ (constant) primer contained a SmaI restriction site that, when ligated to an engineered SrfI site 3′ of the DNA fragment encoding the extracellular domain of GCSFR, was in the same translational reading frame as GCSFR. The resulting PCR fragment was digested with Smal and NotI, gel purified, and ligated into the vector Bluescript-SK (Promega) that had been digested with SmaI and NotI and gel purified. The resulting plasmid was designated pMLK518. Next, a DNA construct encoding a fusion of the IgE C_{H}1-C_{H}4 domains and the extracellular domain of GCSFR was constructed by digesting the pMLK518 plasmid with SmaI and NotI to release the IgE C_{H}1-C_{H}4 DNA fragment. This fragment was ligated into the expression vector pMT21/GCSFR-IgG/Fc that had been digested with SrfI and NotI to release the IgG/Fc sequence. The IgE C_{H}1-C_{H}4 fragment was cloned downstream of the signal sequence and extracellular domain of GCSFR, effectively replacing the IgG/Fc sequence with the IgE constant region sequence. The resulting plasmid was designated pMLK522.

A second chimeric DNA molecule was constructed encoding a fusion protein containing the extracellular domain of GCSFR and the Fc region (C_{H}2-C_{H}4) of the mouse IgE heavy chain. This chimeric DNA molecule was constructed as follows: The IgE Fc region was PCR-amplified using the IgE 3′ primer described above, an IgE 5′ (Fc) primer with the sequence 5′TGTCTAGGCCCGGGCCGACCTGTCAACATCACTGAGCC3′, and the pMLK518 plasmid described above as a PCR template. The IgE 5′ (Fc) primer contained a SmaI restriction site that was compatible with an engineered SrfI site 3′ of the DNA fragment encoding the extracellular domain of GCSFR and that is in the same translational reading frame as GCSFR. The resulting PCR fragment was digested with Smal and Noti and ligated into the pMLK522 described above that had been digested with SrfI/NotI, effectively replacing the C_{H}1-C_{H}4 sequence (the IgE constant region) with the C_{H}2-C_{H}4 sequence (the IgE Fc region). The resulting plasmid was designated pMLK533.

### Example 2

### Transfection of COS7 Cells with Chimeric IgE DNA Constructs

COS7 cells (8×10⁵ cells/plate) were transiently transfected with 5 μg of either the pMLK522 plasmid or the pMLK533 plasmid using the LipofectAMINE (BRL/GIBCO) procedure. Supernatants from the resulting transfectants were analyzed by Western blot using goat anti-IgE polyclonal antibodies (ICN, Catalog #65-369-1) directed against the mouse IgE constant region and determined to secrete recombinant chimeric proteins called GCSFR-IgE (fusion of GCSFR extracellular domain fused to the IgE constant region), or GCSFR-FcE (fusion of GCSFR extracellular domain fused to the IgE Fc region), respectively.

### Example 3

### Dimerization of FcεR1 Receptors Following Cross-Linking of Bound IgE or Chimeric IgE Molecules

The ability of GCSFR-FcE to induce degranulation by standard protocols was evaluated by monitoring the release of hexosaminidase by the rat basophilic leukemia cell line RBL-2H3. Hexosaminidase release can be assayed enzymatically with the chromogenic substrate, p-nitrophenyl-N-acetyl β-glucosaminide (Sigma) (Schwartz, et al., 1979, J. Immunol. 123:1445-50). RBL-2H3 cells were plated at a density of 1×10⁵ cells/well in a 96-well tissue culture plate and incubated overnight at 370° C. The following day, the cells were incubated with varying amounts of IgE (Pharmingen) or GCSFR-FcE (0.001-0.4 μg/ml) for 1 hour at 37° C. In one experiment, the cells were washed and then challenged with either 0.1 μg anti-IgE (Pharmingen) or with 150 ng/ml GCSF (Preprotech) for 1 hour at 37° C. In a second experiment, the cells were incubated with a single, high concentration of IgE (0.1 μg/ml) or GCSFR-FcE (0.1 μg/ml) for 1 hour at 37° C., then washed and challenged with varying concentrations of anti-IgE (0.001-3 μg/ml) or GCSF (0.0007-0.15 μg/ml). In both experiments, the culture media was then removed from the cells and incubated for 1 hour at 37° C. with the hexosaminidase substrate (p-nitro-phenyl-N-acetyl β-D glucosaminide) in a buffer consisting of 25 mM PIPES, 117 mM NaCl, 5 mM KCl, 5.6 mM glucose, 2 mM CaCl₂, 0.8 mM MgCl₂ and 0.1% BSA. The release of p-nitrophenol was measured spectrophotometrically by its absorbance at 400 nm in a 96-well plate reader. The results revealed a dose-dependent increase in the ability of GCSF to induce degranulation of RBL 2H3 cells displaying bound GCSFR-FcE. There was no degranulation observed when either GCSFR-FcE or GCSF were added alone. Similarly, at a single, high concentration of GCSF (0.3 μg/ml), increasing amounts of GCSFR-FcE resulted in a dose-dependent increase in the degree of degranulation as measured by hexosaminidase release. The degree of degranulation observed with GCSFR-FcE and GCSF was comparable to that observed with IgE and anti-IgE.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed:

1. A nucleic acid encoding a chimeric polypeptide molecule comprising an extracellular ligand binding domain of a receptor and an IgE constant region.

2. A nucleic acid encoding a chimeric polypeptide molecule comprising an extracellular ligand binding domain of a receptor and an IgE Fc region.

3. A nucleic acid encoding a chimeric polypeptide molecule comprising a protein interacting region and an IgE constant region.

4. A nucleic acid encoding a chimeric polypeptide molecule comprising a protein interacting region and an IgE Fo region.

5. The nucleic acid molecule of claim 1 or 2 wherein the extracellular ligand binding domain of said receptor is selected from the group consisting of the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, and the EPO receptor extracellular ligand binding domain.

6. The nucleic acid molecule of claim 3 or 4 wherein the protein interacting region is selected from the group consisting of the SH2 domain, the SH3 domain, the PDZ domain, the JAK binding domain, the PH domain, and the WW domain.

7. A chimeric polypeptide molecule comprising an extracellular ligand binding domain of a receptor and an IgE constant region.

8. A chimeric polypeptide molecule comprising an extracellular ligand binding domain of a receptor and an IgE Fc region.

9. A chimeric polypeptide molecule comprising a protein interacting region and an IgE constant region.

10. A chimeric polypeptide molecule comprising a protein interacting region and an IgE Fc region.

11. The chimeric polypeptide molecule of claim 7 or 8 wherein the extracellular ligand binding domain of said receptor is selected from the group consisting of the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, and the EPO receptor extracellular ligand binding domain.

12. The chimeric polypeptide molecule of claim 9 or 10 wherein the protein interacting region is selected from the group consisting of the SH2 domain, the SH3 domain, the PDZ domain, the JAK binding domain, the PH domain, and the WW domain.

13. An assay system comprising
a) a chimeric polypeptide molecule comprising an extracellular ligand binding domain of a receptor fused to an IgE constant region; and
b) a means of detecting or measuring ligand binding to the chimeric polypeptide molecule.

14. An assay system comprising
a) a chimeric polypeptide molecule comprising an extracellular ligand binding domain of a receptor fused to an IgE Fc region; and
b) a means of detecting or measuring ligand binding to the chimeric polypeptide molecule.

15. An assay system comprising
a) a chimeric polypeptide molecule comprising a protein interacting region fused to an IgE constant region; and
b) a means of detecting or measuring ligand binding to the chimeric polypeptide molecule.

16. An assay system comprising
a) a chimeric polypeptide molecule comprising a protein interacting region fused to an IgE Fc region; and
b) a means of detecting or measuring ligand binding to the chimeric polypeptide molecule.

17. The assay system of any one of claims 13-16 wherein said means of detecting or measuring ligand binding to said chimeric polypeptide molecule comprises a means of detecting or measuring dimerization of a complex of the chimeric polypeptide molecule and a cell surface FcεR1 molecule, wherein the dimerization results from ligand binding to said chimeric polypeptide molecule.

18. The assay system of claim 17 wherein said means of detecting or measuring dimerization of said complex comprises detecting or measuring cellular degranulation, wherein degranulation results from dimerization.

19. The assay system of claim 18 wherein said means of measuring said cellular degranulation is selected from the group consisting of a calorimetric assay, a radioisotopic assay, and a fluorescence assay.

20. The assay system of any one of claims 13 or 14 wherein said extracellular ligand binding domain of said receptor is selected from the group consisting of the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, and the EPO receptor extracellular ligand binding domain.

21. The assay system of claims 15 or 16 wherein said ligand is selected from the group consisting of a protein, peptide, lipid, carbohydrate, nucleic acid, and a small molecule.

22. A cell line stably expressing a chimeric polypeptide molecule comprising an extracellular ligand binding domain of a receptor and an IgE constant region.

23. A cell line stably expressing a chimeric polypeptide molecule comprising an extracellular ligand binding domain of a receptor and an IgE Fc region.

24. A cell line stably expressing a chimeric polypeptide molecule comprising a protein interacting region and an IgE constant region.

25. A cell line stably expressing a chimeric polypeptide molecule comprising a protein interacting region and an IgE Fc region.

26. The cell line of claims 22 or 23 wherein said extracellular ligand binding domain of a receptor is selected from the group consisting of the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, and the EPO receptor extracellular ligand binding domain.

27. The cell line of claims 24 or 25 wherein said protein interacting region is selected from the group consisting of the SH2 domain, the SH3 domain, the PDZ domain, the JAK binding domain, the PH domain, and the WW domain.

28. A method of identifying a ligand for a receptor comprising:
(a) contacting a cell expressing a cell surface FcεR1 molecule with a chimeric polypeptide molecule comprising an extracellular ligand binding domain of a receptor and an IgE constant region, under conditions whereby said chimeric polypeptide molecule binds to said FcεR1 molecule to form a complex;
(b) contacting said cell bearing said complex with a ligand; and
(c) detecting or measuring said ligand binding to said complex.

29. The method according to claim 28 wherein ligand binding is detected or measured by detecting or measuring dimerization of the complexes, wherein dimerization is indicative of ligand binding.

30. A method of identifying a ligand for a receptor comprising:
(a) contacting a cell expressing a cell surface FcεR1 molecule with a chimeric polypeptide molecule comprising an extracellular ligand binding domain of a receptor and an IgE Fc region, under conditions whereby said chimeric polypeptide molecule binds to said FcεR1 molecule to form a complex;
(b) contacting said cell bearing said complex with a ligand; and
(c) detecting or measuring said ligand binding to said complex.

31. The method according to claim 30 wherein ligand binding is detected or measured by detecting or measuring dimerization of the complex, wherein dimerization is indicative of ligand binding.

32. The method of claim 29 or 30 wherein said cell expressing a cell surface FcεR1 molecule is a mast cell or a basophil.

33. The method of claim 29 or 30 wherein said means of detecting or measuring dimerization of said complex comprises detecting or measuring cellular degranulation resulting from dimerization.

34. The method of claim 33 wherein said means of measuring said cellular degranulation is selected from the group consisting of a calorimetric assay, a radioisotopic assay, and a fluorescence assay.

35. The method of claim 29 or 30 wherein said extracellular ligand binding domain of said receptor is selected from a group consisting of the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, and the EPO receptor extracellular ligand binding domain.

36. The method of claim 29 or 30 wherein said ligand is selected from the group consisting of a protein, peptide, lipid, carbohydrate, nucleic acid, and a small molecule.

37. A method of identifying a candidate antagonist to a receptor comprising:
(a) contacting a cell expressing a cell surface FcεR1 molecule with:
(i) a chimeric polypeptide molecule comprising an extracellular ligand binding domain of a receptor and the IgE constant region; and
(ii) a ligand for said receptor, in the presence and absence of a candidate antagonist for said receptor, and under conditions whereby said chimeric polypeptide molecule binds to said cell surface FcεR1 molecule to provide a cell bearing complexes of said FcεR1 molecule and said chimeric polypeptide molecule; and
(b) measuring binding of said ligand to said complex in the presence and absence of said candidate antagonist, wherein decreased ligand binding in the presence of said candidate antagonist is indicative of identification of an antagonist.

38. A method of identifying a candidate antagonist to a receptor comprising:
(a) contacting a cell expressing a cell surface FcεR1 molecule with:
(i) a chimeric polypeptide molecule comprising an extracellular ligand binding domain of a receptor and the IgE Fc region; and
(ii) a ligand for said receptor, in the presence and absence of a candidate antagonist for said receptor, and under conditions whereby said chimeric polypeptide molecule binds to said cell surface FcεR1 molecule to provide a cell bearing complexes of said FcεR1 molecule and said chimeric polypeptide molecule; and
(b) measuring binding of said ligand to said complex in the presence and absence of said candidate antagonist, wherein decreased ligand binding in the presence of said candidate antagonist is indicative of identification of an antagonist.

39. The method of claim 37 or 38 wherein said cell expressing a cell surface FcεR1 molecule is a mast cell or a basophil.

40. The method of claim 37 or 38 wherein said means of measuring dimerization of said complex comprises measuring cellular degranulation resulting from dimerization.

41. The method of claim 40 wherein said means of measuring said cellular degranulation is selected from the group consisting of a calorimetric assay, a radioisotopic assay, and a fluorescence assay.

42. The method of claim 37 or 38 wherein said extracellular ligand binding domain of said receptor is selected from a group consisting of the GCSF receptor extracellular ligand binding domain, the MuSK receptor extracellular ligand binding domain, the BMP receptor extracellular ligand binding domain, the OB receptor extracellular ligand binding domain, the CNTFRα extracellular ligand binding domain, the gp130 receptor extracellular ligand binding domain, and the EPO receptor extracellular ligand binding domain.

43. The method of claim 37 or 38 wherein said ligand is selected from the group consisting of a protein, peptide, lipid, carbohydrate, nucleic acid, and a small molecule.

44. A method of making a chimeric polypeptide molecule comprising the extracellular binding domain of a receptor and the IgE constant region comprising:
(a) introducing the nucleic acid of claim 1 into a host cell;
(b) maintaining said host cell under conditions whereby said nucleic acid is expressed to provide a chimeric polypeptide molecule; and
(c) recovering said chimeric polypeptide molecule.

45. A method of making a chimeric polypeptide molecule comprising the extracellular binding domain of a receptor and the IgE Fc region comprising:
(a) introducing the nucleic acid of claim 2 into a host cell;
(b) maintaining said host cell under conditions whereby said nucleic acid is expressed to provide a chimeric polypeptide molecule; and
(c) recovering said chimeric polypeptide molecule.

46. A method of making a chimeric polypeptide molecule comprising a protein interacting region and the IgE constant region comprising:
(a) introducing the nucleic acid of claim 3 into a host cell;
(b) maintaining said host cell under conditions whereby said nucleic acid is expressed to provide a chimeric polypeptide molecule; and
(c) recovering said chimeric polypeptide molecule.

47. A method of making a chimeric polypeptide molecule comprising a protein interacting region and the IgE Fc region comprising:
(a) introducing the nucleic acid of claim 4 into a host cell;
(b) maintaining said host cell under conditions whereby said nucleic acid is expressed to provide a chimeric polypeptide molecule; and
(c) recovering said chimeric polypeptide molecule.

48. The method of any one of claims 44-47 wherein said host cell is selected from the group consisting of a bacterial cell, a yeast cell, an insect cell, or a mammalian cell.

49. A vector comprising the nucleic acid of claim 1.

50. A vector comprising the nucleic acid of claim 2.

51. A vector comprising the nucleic acid of claim 3.

52. A vector comprising the nucleic acid of claim 4.

53. A vector comprising the nucleic acid of claim 5.

54. A vector comprising the nucleic acid of claim 6.
